(19) United States
(12) Patent Application Publication
(10) Pub. No.: US 2018/0311162 A1
(43) Pub. Date: Nov. 1, 2018

(54) SKIN BARRIER ENHANCING ARTICLE AND MANUFACTURING METHOD

(71) Applicant: SHEN WEI (USA) INC., Union City, CA (US)

(72) Inventors: Belle L. Chou, Union City, CA (US); Sisitha Sudarshana, Homagama (LK); Athawuda Arachchige Ranthi Manahari Dias, Athurugiriya (LK)

(21) Appl. No.: 15/583,911

(22) Filed: May 1, 2017

(51) Int. Cl. A61K 9/127; A61K 31/4172; A61K 9/00; A61K 31/20

(52) U.S. Cl. CPC A61K 9/127; A61K 31/20; A61K 9/0014; A61K 31/4172

(57) ABSTRACT

The present invention relates to developing a skin pH balancing natural composition coating applied to the inside surface of an elastomeric flexible article, such as single use disposable glove. The skin pH balancing natural composition coating comprises a mixture of amino acids encapsulated in liposomes and monovalent cations encapsulated in liposomes and free fatty acids which enhance the skin barrier function through skin pH balancing. The present invention also relates to the method of making the skin pH balancing natural composition coating and the method of applying the coating to the inside surface of an elastomeric flexible article, such as a single use disposable glove.

### BACKGROUND OF THE INVENTION

### 1. Field of the Invention

The exemplary embodiment(s) of the present invention generally relates to an elastomeric flexible article for application to a person's skin. More specifically, the exemplary embodiment(s) of the present invention relates to a skin barrier enhancing coating for an inside surface of an article, in particular a glove, with a skin pH lowering natural composition.

### 2. Background

A principal problem is higher vulnerability to skin disease when the skin barrier is depleted. Occupational dermatitis is a common type of skin disease spreading in many industries worldwide. Irritant contact dermatitis and allergic contact dermatitis are the highest rated diseases in the industries. Disruption of natural skin barrier function, due to chemical contact and frequent use of soaps, hand washers, alcoholic sanitizers and detergents result in removal of epidermal barrier lipids, and natural moisturizing factors (NMF), which lead to loss of skin moisture and skin pH alterations (from acidic to basic), and leaving skin unprotected to external allergens.

The skin barrier is derived by anucleate, cornified, outermost layers of the epidermis, collectively known as the stratum corneum. It is known that the stratum corneum is composed of a mixture of lipids and proteins, such as ceramides, cholesterols, free fatty acids, filaggrin and keratin respectively.

Skin pH is one key factor involved in skin barrier homeostasis. Several physiological mechanisms contribute to the formation of the acid mantel resulting in the formation of a skin barrier at an acidic pH. The proposed physiological mechanisms for the stratum corneum acidification describes: (1) Phospholipids to free fatty acids (FFA) pathway; (2) Na⁺/H⁺ exchange (NHE1); (3) Histidine to trans-Urocanic acid (UCA) pathway; and (4) Sweat and sebum gland secretions. Formation of free fatty acids, trans-Urocanic acid, and monovalent ion exchange, contributes to pH reduction in the stratum corneum, and maintaining the desired skin pH at 5-5.5. This pH is critical for a healthy skin barrier, retards the entry of pathogenic microorganisms through the skin, and prevents the occurrence of skin diseases.

During the epidermal differentiation, free fatty acids particularly, medium and long chain fatty acids are derived from lamellar-phospholipids by an enzymatic degradation and exists in the extracellular spaces in between the corneocytes maintaining the pH of the stratum corneum. Apart from this function, free fatty acids also contribute to the flexibility of the stratum corneum.

NHE1 is a transport protein of the monovalent cations in keratinocyte cell membranes and has been shown to regulate intracellular pH by electroneutral exchange of extracellular Na⁺ ion for intracellular H⁺ ion, thereby creating an acidic pH in the border between stratum granulosum and stratum corneum. This facilitates the ceramide lipid formation by acid sphingomyelinase and β-glucocerebrosidase, thus enhancing the skin barrier function through a healthy lipid layer.

During epidermal differentiation, Histidine amino acid, resulting from Filaggrin proteolysis, is further converted into trans-Urocanic acid, by catalysis of histidase enzyme, in corneocytes. trans-Urocanic acid, is another important factor which regulates the stratum corneum pH.

Sweat and sebum gland secretes lactic acid and lipids on to the outer skin surface, where the lipids are further converted to free fatty acids by the action of skin microflora, thus contributing to maintain an acidic skin pH.

A solution to skin barrier depletion resulting in higher vulnerability to skin disease has been to coat the inside of gloves with natural ingredients. However, these natural ingredients are non-selective. These natural ingredients also do not actively regulate skin barrier biological mechanisms to determine the pH of the stratum corneum. Furthermore, most of the prior solutions are hydrophilic in nature making it difficult to penetrate through the hydrophobic epidermis layer of the skin. This further reduces the desired effects of such natural ingredients as they are not penetrating into the active sites inside the layers of the skin.

Another solution to this problem is aloe coated gloves; however, the aloe is simply for moisturizing the skin and is not actively regulating the pH level of the skin. As such, the prior solutions have failed to provide an effective and efficient means of regulating skin pH to an optimum level for enhancing the barrier function of the skin.

Accordingly, there is a need for a coating that can be applied to an elastomeric article, such as a glove, the coating comprising a mixture of active ingredients that can actively regulate skin pH to enhance the barrier function of the skin.

### SUMMARY

According to an embodiment of the present invention, there is an elastomeric article having an inside surface containing a coating of a skin pH balancing natural composition comprising a dried solution of active ingredients including liposome encapsulated amino acids, liposome encapsulated monovalent cations, and free fatty acids.

According to another embodiment of the present invention, the dried solution on the elastomeric article is dissolvable on skin and the liposomes enhance absorption of the active ingredients into the skin. In a further embodiment, the active ingredients reduce skin pH by 0.2-0.5 units.

According to yet another embodiment of the present invention, there is an elastomeric, fluid impermeable glove comprising a skin pH balancing composition coated and dried on an inside surface of the glove. The skin pH balancing composition comprises: a plurality of active ingredients comprising amino acids, free fatty acids, and monovalent cations where the amino acid and the monovalent cations are encapsulated in liposomes. The molar ratio of the free fatty acids to the amino acids to the monovalent cations to the liposomes is preferably 2:3:1:1 and the active ingredients reduce skin pH by 0.2-0.5 units.

According to yet another embodiment of the present invention, there is a method of manufacturing a fluid impermeable elastomeric article comprising a skin pH balancing composition. The method, according to an embodiment, comprises preparing a skin pH balancing composition including encapsulating amino acids within liposomes and encapsulating monovalent cations within liposomes and mixing the liposome encapsulated amino acids and liposome encapsulated monovalent cations, with a free fatty acid, water, and an emulsifying agent; and applying the skin pH balancing composition to an inside surface of the article.

These features, advantages and other embodiments of the present invention are further made apparent, in the remainder of the present document, to those of ordinary skill in the art.

### BRIEF DESCRIPTION OF THE DRAWINGS

In order to more fully describe embodiments of the present invention, reference is made to the accompanying drawings. These drawings are not to be considered limitations in the scope of the invention, but are merely illustrative.

FIG. 1 illustrates a cross sectional view of an elastomeric article comprising a coating containing active ingredients on an inside surface of the article in contact with a user's skin, according to an embodiment of the present invention.

FIG. 2 is a detail view taken from FIG. 1 illustrating the active ingredients entering the skin, according to an embodiment of the present invention.

FIG. 3 illustrates the target sites of the active ingredients, which comprise free fatty acids, monovalent cations, and amino acids in the epidermis, according to an embodiment of the present invention.

FIG. 4 illustrates a liposome surrounding monovalent cations, according to an embodiment of the present invention.

FIG. 5 illustrates a liposome surrounding hydrophilic amino acids, according to an embodiment of the present invention.

FIG. 6 illustrates the penetration of monovalent cations and amino acids surrounded by liposomes in the stratum corneum, according to an embodiment of the present invention.

FIG. 7 illustrates a liposome releasing encapsulated amino acids in the stratum corneum, according to an embodiment of the present invention.

FIG. 8 illustrates a liposome releasing encapsulated monovalent cations at the interface between the stratum granulosum and the stratum corneum, according to an embodiment of the present invention.

FIG. 9 illustrates a method of making an elastomeric article having a preparation of a skin pH balancing natural composition including a method of processing the active ingredients in the preparation applied to the article, according to an embodiment of the present invention.

### DETAILED DESCRIPTION OF SPECIFIC EMBODIMENTS

The description above and below and the drawings of the present document focus on one or more currently preferred embodiments of the present invention and also describe some exemplary optional features and/or alternative embodiments of the present invention. The description and drawings are for the purpose of illustration and not limitation. Those of ordinary skill in the art would recognize variations, modifications, and alternatives. Such variations, modifications, and alternatives are also within the scope of the present invention. Section titles are terse and are for convenience only.

Throughout the description and drawings, example embodiments of the present invention are given with reference to specific configurations. It will be appreciated by those of ordinary skill in the art that the present invention can be embodied in other specific forms. Those of ordinary skill in the art would be able to practice such other embodiments of the present invention without undue experimentation. The scope of the present invention, for the purpose of the present patent document, is not limited merely to the specific example embodiments of the present invention or alternatives of the foregoing description.

An embodiment of the present invention is an elastomeric article having an inside surface 3 and an outside surface 4. The inside surface 3 can also be referred to as the inner surface and the inner surface/inside surface 3 is the skin-facing or skin contacting surface of the wearer/user when the article is worn on in use. The elastomeric flexible article can be a glove, such as a disposable, fluid impermeable examination type glove or disposable protective glove 2. An elastomeric flexible article, according to some embodiments of the present invention is a disposable protective glove, but other forms of articles may also be used, such as protective articles worn on, or to cover, a portion of the skin. The elastomeric flexible article can be made of a material selected from nitrile butadiene latex, natural rubber latex, polychloroprene, polyurethane, polyisoprene, PVA, acrylic, butyl, silicone rubber, fluoro-elastomer, and PVC, or combinations thereof, where the inside surface 3 of the glove 2 is coated with a mixture of natural ingredients facilitating the reduction of skin pH.

In one embodiment of the present invention, the inside surface 3 of the disposable glove 2 can contain a coating 6 comprising a mixture of natural active ingredients extracted from plant based materials. The active ingredients can be made from a combination of medium fatty acids (MFA), long fatty acids (LFA), rich natural oils, amino acids, for example, histidine, and sodium and potassium monovalent ions. The mixture of medium fatty acids and long fatty acids may include lauric acid, palmitic acid, stearic acid, oleic acid, linoleic acid, alpha-linolenic acid, and combinations thereof. These ingredients can be selected from one or more of the following sources listed in Table 1.

TABLE 1
Natural sources of the active ingredients

| | |
|---|---|
| A-Amino Acids | aloe vera/hydrolyzed soy protein/hydrolyzed |
| | wheat protein and red/brown seaweeds. |
| B-Free Fatty Acids | avocado oil/shea butter/coconut oil/olive oil/ |
| | corn oil |
| C-Monovalent Cations | banana powder and grape seeds extracts |
| D-Liposomes | soy beans, sunflower seed |

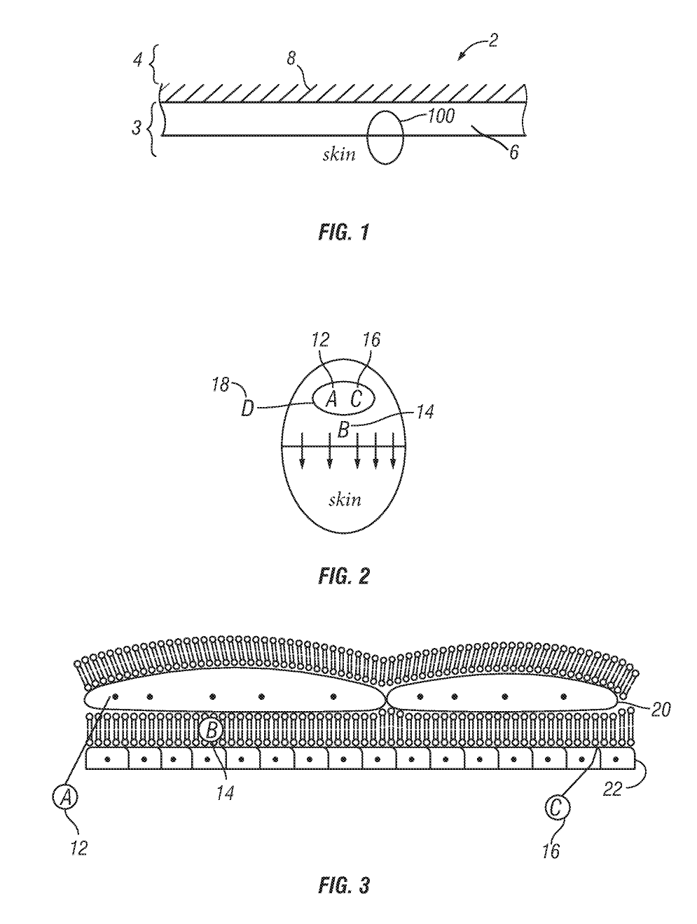

FIG. 1 illustrates a cross sectional view of an article, such as a glove 2, in contact with a user's skin, the glove 2 comprises a coating 6 comprising active ingredients, according to an embodiment of the present invention. The cross sectional view shows the article having an indeterminate or variable length and thickness as well as a magnified view of the coating thickness for reference purposes. The glove 2 comprises an elastomeric layer 8 having an inside surface 3 that faces the user's skin and an outside surface 4 that faces the external environment. The coating 6 is applied to the inside surface 3 as a dried coating and comes in contact with the skin of a user. Once the glove 2 is worn and comes in direct contact with the user's skin, the coating 6 dissolves due to the warm and moist environment created inside the glove 2. This facilitates the active ingredients, particularly B-free fatty acids 14, A-amino acids 12 and C-monovalent cations 16 to absorb into the skin and regulate the natural skin's pH for a healthy skin barrier (not shown, see FIG. 2). The concentration of the active ingredients of the coating 6 can range from 5-16%, more preferably 10-12% with a ratio of free fatty acids: amino acids: monovalent cations: liposomes to approximately 2:3:1:1.

FIG. 2 is a detailed view of FIG. 1 at 100, illustrating the active ingredients, A-amino acids 12, B-free fatty acids 14, C-monovalent cations 16, D-liposomes 18, according to an embodiment of the present invention. The coating 6 comprising the active ingredients are absorbed into the user's outer skin layer, the stratum corneum 20 in the epidermis. The A-amino acids 12 and C-monovalent cations 16 are encapsulated within D-liposomes 18, which can penetrate into the user's skin. The liposomes enhance the absorption of the active ingredients into the skin. The D-liposomes 18 can be phosphatidylcholines extracted from plants. The epidermis comprises the: stratum basale, stratum spinosum, stratum granulosum 22, stratum corneum 20. The stratum corneum 20 is the target site of the active ingredients in the present invention.

FIG. 3 illustrates the stratum corneum 20 and stratum granulosum 22, which can be target sites of B-free fatty acids 14, C-monovalent cations 16, and A-amino acids 12 in the epidermis, according to an embodiment of the present invention. As illustrated in FIG. 3, B-free fatty acids 14 particularly, medium chain fatty acids and long chain fatty acids in the coating 6 penetrate the epidermis of the skin through the intercellular spaces of the stratum corneum 20. The lower pH of the B-free fatty acids 14 reduces the overall pH of the stratum corneum 20. FIG. 3 also illustrates C-monovalent cations 16 such as Na⁺ ions in the extracellular space exchanging with intracellular H+ ion by the transport channel called NHE1 located in the cell lining of keratinocyte cell membranes. C-monovalent cations such as H⁺ ions can also be used. The sodium-hydrogen antiporter 1 (NHE1), which is a transport protein of C-monovalent cation 16 in keratinocyte cell membranes has been shown to regulate intracellular pH by electroneutral exchange of extracellular Na⁺ ion for intracellular H⁺ ion, creating an acidic pH in the border between the stratum granulosum 22 and stratum corneum 20. This facilitates the ceramide lipid formation by the low pH dependent enzymes (i.e. acid sphingomyelinase and β-glucocerebrosidase), thus enhancing the skin barrier function through a dense lipid layer.

FIG. 3 also illustrates A-amino acids 12, particularly Histidine, which converts into trans-Urocanic acid, by catalysis of the histidase enzyme, in the cells of the stratum corneum 20. trans-Urocanic acid, is another important factor in regulating the pH of stratum corneum 20.

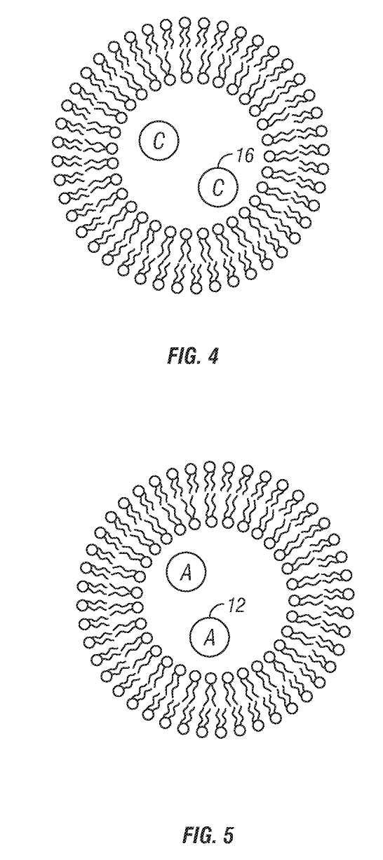

FIG. 4 illustrates C-monovalent cations 16 encapsulated inside a hydrophobic carrier called a D-liposome 18. D-liposome 18 facilitates the hydrophilic C-monovalent cations 16 penetration into the target site, which is the interface between the stratum granulosum 22 and the stratum corneum 20. FIG. 5 illustrates hydrophilic A-amino acids 12 encapsulated inside a hydrophobic carrier called a D-liposome 18, ready to penetrate through the hydrophobic intercellular route of the stratum corneum 20.

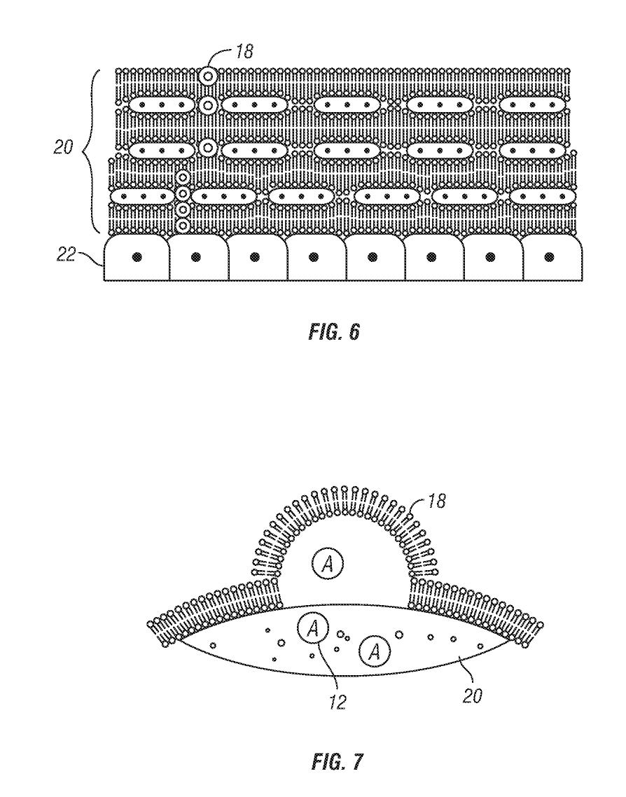

FIG. 6 illustrates the penetration of monovalent cations and amino acids surrounded by liposomes 18 in the stratum corneum 20, according to an embodiment of the present invention. The liposomes 18 encapsulating the A-amino acids 12 target the stratum corneum 20 and the liposomes 18 encapsulating the C-monovalent cations 16 target the interface between the stratum granulosum 22 and the stratum corneum 20.

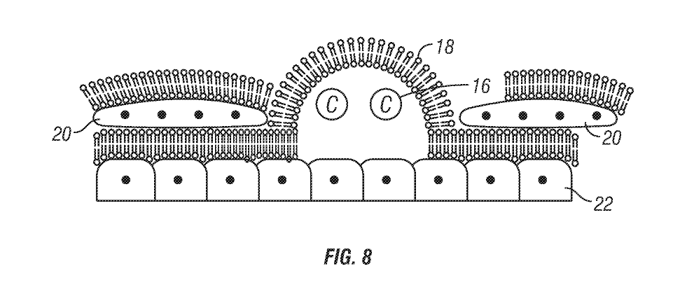

FIG. 7 illustrates the D-liposomes 18 releasing encapsulated A-amino acids 12 in the stratum corneum 20, according to an embodiment of the present invention. This is done when the D-liposomes 18 dissolve in the intercellular lipids of the stratum corneum 20. The chemical and physical structures of these liposomes are similar to that of the lipid membrane in the skin barrier, and so offer additional protection to the skin.

FIG. 8 illustrates liposomes 18 releasing encapsulated C-monovalent cations 16 at the interface between the stratum granulosum 22 and the stratum corneum 20, according to an embodiment of the present invention. This is done when the D-liposomes 18 dissolve in the intercellular lipids of the stratum corneum 20.

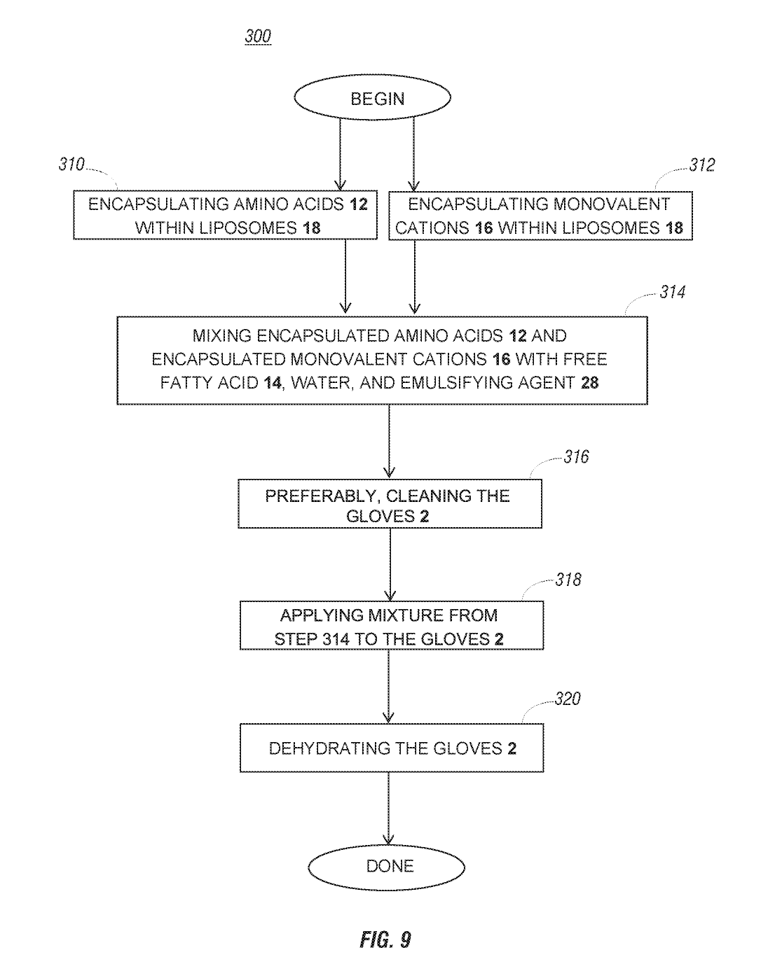

FIG. 9 illustrates one method 300 of processing the active ingredients and its application to an elastomeric article, according to an embodiment of the present invention. A-amino acids 12 are encapsulated within D-liposomes 18 (step 310) and C-monovalent cations 16 are encapsulated within D-liposomes 18 (step 312). In performing the above encapsulation, the rate of adding A-amino acids 12 and C-monovalent cations 16 into D-liposomes 18 is kept at about 50 mL per minute with a reaction temperature in the range of 40° C.-50° C., at a mixing speed of about 500-600 rounds per minute. The process of encapsulation is continued for 30-40 minutes.

The above mentioned encapsulated A-amino acids 12 and C-monovalent cations 16 are mixed with a free fatty acid source and water. Additionally, this step can be done in the presence of an emulsifying agent 28 (step 314). The B-free fatty acids 14 can be selected from a group listed in Table 1 and a likely material. To attain a homogeneous mixture, hydrophilic and hydrophobic ingredients in the active ingredient mixture are mixed together with an emulsifying agent 28, which provides a fundamental outcome for a uniform coating. The emulsifying agent 28 can be selected from the following range or as appropriate gum arabic, soya lecithin, and bee wax. In preparing the final active ingredient mixture above, the mixing is carried out at preferably 1000-1500 rounds per minute, for duration of preferably up to 60 minutes. In an embodiment, the duration of mixing the encapsulated amino acids 12 and the encapsulated monovalent cations 16 is about 45 to 60 minutes. The following formulas in Table 2 illustrate different compositions related to embodiments of the present invention. Formulas I and II have a lower drying temperature leaving no residues, and no stickiness in the inside surface 3 of the glove 2, and the ease of coating is much better compared to Formula III. Formulas I and II were selected for further proceedings based on ease of processing.

TABLE 2
Coating Composition
Coating Composition (wet weight %)

| | Formula I | Formula II | Formula III |
|---|---|---|---|
| | (5%) | (12%) | (16%) |
| A-Amino Acids | 1.5 | 3.0 | 3.5 |
| B-Free Fatty Acids | 2.0 | 4.5 | 4.5 |
| C-Monovalent Cations | 0.5 | 1.5 | 2.5 |
| D-Liposomes | 0.7 | 1.5 | 3.5 |
| Emulsifying agent | 1.0 | 1.5 | 2.0 |
| Water | 94.3 | 88 | 84 |

The application of the prepared mixture to gloves preferably begins with gloves that are clean and free of protein residue, powder, or other surface contaminants. Therefore, the step 316 is preferably included in the method 300 to remove such contaminants. The prepared mixture is applied to the gloves 2 (step 318), for example, by means of spraying the mixture on to the inside surface 3 of the glove 2. Alternatively, gloves 2 can be immersed into a solution containing this active ingredient mixture. In the latter method, the gloves are immersed in the mixture for at least 2 minutes to allow the mixture to absorb onto the inside surface 3.

Active ingredients are attached to the inside surface 3 of the glove 2 through a controlled dehydration process (step 320). Water in the solution mixture is caused to evaporate through hot air drying. Coated gloves 2 are dried in a pre-heated oven at 45° C.-55° C. more preferably at 50° C., and the drying process continues to about 30-40 minutes. The temperature of the hot air is thoroughly maintained at the above range to avoid loss of active ingredients in the coating 6. Alternatively, steps 318 and 320 can be accomplished by a hot air oven with a device to spray and tumble simultaneously during drying to distribute the mixture of natural ingredients evenly on the inside surface 3 of glove 2 to form a uniform coating 6. After the dehydration process, a resulting dried solution of active ingredients adheres to the inside surface of the glove 2 which is dissolvable on skin under the warm and moist environment created inside the glove 2 during use.

The use of a combination of natural extracts within the scope of this invention, will effectively improve skin barrier function or enhance its recovery rate by reducing skin pH by 0.2-0.5 units. The measurement of skin pH following treatment with formulas I and II, in accordance with the invention, is summarized below.

60 female subjects between the ages of 30-40, were selected from a factory setting, where their routine work involves wearing gloves. Subjects were placed in two groups of 30 each. Each group's skin pH was examined for 30 days following use of the coated glove. Gloves were prepared with the coating 6 as per the Formula I and II separately and distributed among the two groups. Subjects were advised to avoid using any topical applications on the skin during the trial and instructed to wear the coated gloves on both hands. Skin pH measurements were recorded after completion of eight hours of wearing gloves. Measurements were taken using skin pH probe 905 (Courage+Khazaka electronic GmbH). The mean values of skin pH are shown in Table 3 below. The results show that the Formula II coated glove has a significant skin pH reduction capacity than the coating of Formula I.

TABLE 3
Mean Skin pH (baseline vs. coated glove)

| Group-1 Formula I | | Group-2 Formula II | |
|---|---|---|---|
| Baseline | Coated | Baseline | Coated |
| 5.5 | 5.5 | 5.5 | 5.1 |

While particular embodiments of the present invention have been shown and described, it will be obvious to those of skills in the art that based upon the teachings herein, changes and modifications may be made without departing from this exemplary embodiment(s) of the present invention and its broader aspects. Therefore, the appended claims are intended to encompass within their scope all such changes and modifications as are within the true spirit and scope of this exemplary embodiment(s) of the present invention.

1. An elastomeric article having an inside surface containing a coating of a skin pH balancing natural composition comprising:
a dried solution of active ingredients including liposome encapsulated amino acids, liposome encapsulated monovalent cations, and free fatty acids,
wherein the dried solution is dissolvable on skin, and
the liposomes enhance absorption of the active ingredients into the skin.

2. The elastomeric article of claim 1, wherein each liposome is a phosphatidylcholine extracted from a plant.

3. The elastomeric article of claim 1, where the amino acids are from a natural plant extract selected from the group consisting of aloe vera, hydrolyzed soy protein, hydrolyzed wheat protein, red seaweed, brown seaweed, and combinations thereof.

4. The elastomeric article of claim 3, where the amino acids are an L-histidine.

5. The elastomeric article of claim 1, where the free fatty acids are selected from the group consisting of avocado oil, shea butter, coconut oil, olive oil, and corn oil, and combinations thereof.

6. The elastomeric article of claim 5, where the free fatty acids are a mixture of medium fatty acids and long fatty acids selected from the group consisting of lauric acid, palmitic acid, stearic acid, oleic acid, linoleic acid, alpha-linolenic acid, and combinations thereof.

7. The elastomeric article of claim 1, where the monovalent cations are selected from a group consisting of dried banana powder, grape seeds, and combinations thereof.

8. The elastomeric article of claim 7, where the monovalent cations are sodium and potassium.

9. The elastomeric article of claim 1, where a molar ratio of the free fatty acids to amino acids to the monovalent cations, to the liposomes is preferably 2:3:1:1.

10. The elastomeric article of claim 1, wherein
the active ingredients reduce skin pH by 0.2-0.5 units.

11. The elastomeric article of claim 1, comprising a fluid impermeable glove wherein the glove is made of a layer of material selected from the group consisting of nitrile butadiene latex, natural rubber latex, polychloroprene, polyurethane, polyisoprene, PVA, acrylic, butyl, silicone rubber, fluoro-elastomer, and PVC.

12. A method of manufacturing an elastomeric article comprising a skin pH balancing composition that enhances absorption of active ingredients into the skin, the method comprising the steps of:
a) preparing a skin pH balancing composition including encapsulating amino acids within liposomes and encapsulating monovalent cations within liposomes; and
mixing the liposome encapsulated amino acids and liposome encapsulated monovalent cations, with a free fatty acid, water, and an emulsifying agent;
b) applying the skin pH balancing composition to an inside surface of the article; and
c) drying the composition.

13. The method of claim 12, wherein a rate of encapsulation is about 50 mL/minute.

14. The method of claim 12, wherein a temperature of encapsulation is in a range of about 40° C.-50° C.

15. The method of claim 12, wherein a mixing speed of encapsulation is about 500-600 rounds per minute.

16. The method of claim 12, wherein a duration of encapsulation is about 30-40 minutes.

17. The method of claim 12, wherein a speed of mixing the liposome encapsulated amino acids and the liposome encapsulated monovalent cations is about 1000-1500 rounds per minute.

18. The method of claim 12, wherein a duration of mixing the liposome encapsulated amino acids and the liposome encapsulated monovalent cations is about 45-60 minutes.

19. The method of claim 12, wherein applying the skin pH balancing composition comprises spraying the composition onto the inside surface of the article.

20. The method of claim 12, wherein applying the skin pH balancing composition comprises immersing the inside surface of the article into the composition.
